(19) United States
(12) Patent Application Publication
(10) Pub. No.: US 2018/0321213 A1
(43) Pub. Date: Nov. 8, 2018

(54) METHOD FOR MONITORING CHEMICAL PARAMETERS FROM AN OPERATING MINERAL OR WATER PROCESSING PLANT; SYSTEM THEREFOR; PROCESSING PLANT COMPRISING SUCH SYSTEM

(71) Applicant: Magotteaux International S.A., Vaux-sous-Chèvremont (BE)

(72) Inventor: Christopher John GREET, Vaux-sous-Chèvremont (BE)

(73) Assignee: Magotteaux International S.A., Vaux-sous-Chèvremont (BE)

(21) Appl. No.: 15/774,408

(22) PCT Filed: Nov. 17, 2016
(86) PCT No.: PCT/EP2016/078008

§ 371 Date: May 8, 2018

(30) Foreign Application Priority Data
Nov. 17, 2015 (BE) BE2015/5752

(51) Int. Cl. G01N 33/18; G01N 33/24

(52) U.S. Cl. CPC G01N 33/18; G01N 33/24; G01N 33/1806

(57) ABSTRACT

System for monitoring pulp chemistry data of an operating mineral or water processing plant, comprising at least one sample point on a process stream of the operating plant for continuously sampling slurry from the process stream, a sample chamber for receiving the sampled slurry and a feed line between the sample point and the sample chamber to feed the sampled slurry to the sample chamber, wherein the sample chamber is located on the plant site and is arranged for measuring pulp chemistry data of the sampled slurry, further comprising a control system for processing the measured data and providing the measured data to an operator interface element in real-time.

The invention relates to a method for measuring and/or monitoring chemical parameters in a processing plant, such as a mineral processing plant or a water treatment plant.

The processes in a mineral processing plant are often difficult to control as many of the parameters affecting the mineral separations are not measured, therefore the process sometimes becomes unstable for no apparent reason. Many of the methods currently available that might provide the operator with some indication of what process parameters have changed generally require that a representative sample be taken from the appropriate process stream(s) and analyzed ex-situ from the process. The delay in receiving the data from the external analysis means that solutions are often applied retrospectively, and often inappropriately as the condition no longer exists. Because of this, the cost of obtaining this data is often viewed as excessive or prohibitive, so the data may not be collected at all. Both approaches invariably lead to poor decision making and losses in metal production. Similar problems occur in a waste water treatment plant, wherein waste water is understood to be a natural run off, tailing dam water, sewage, grey water etc.

It is known from laboratory experiments that the correct measurement of pulp chemical parameters (such as pH, pulp potential (ORP or Eh), dissolved oxygen, temperature, conductivity, oxygen demand and EDTA extractable metal ion analysis) can provide valuable information about changes in the mineralogy of the ore feed to the process. These parameters can also provide important data that may affect the metallurgical performance (i.e. concentrate grade and mineral recovery) of the process. It is felt that if the pulp chemical data was available to the operator of mineral processing plants it may benefit the management of the process, which may improve the metallurgical performance.

It is an object of the invention to provide a method for measuring and/or monitoring pulp chemistry of an operating mineral processing plant that obviates at least one of the above mentioned drawbacks.

Thereto, a method is provided according to claim 1.

By continuously sampling a flow of slurry from a process stream within the operating mineral processing plant and filling a sample chamber on the plant site therewith, the sampled slurry can be measured and/or analyzed on the plant site itself. So, there is a reduced need to collect sample and bring the sample ex-situ to an external laboratory, analyze the sample there and bring the analyzed data back to the plant a few days later. Analyzing the sampled slurry and measuring the pulp chemistry data thereof in-situ, brings a major advantage to the plant operator.

Surprisingly, the inventor found that the same parameters give valuable information about the performance of water treatment processes. Therefore, the invention is equally well suitable for a water processing plant, such as a water treatment plant. In this specification, the terminology such as pulp or slurry is equally used for referring to a mineral process stream as to a water process stream.

Advantageously, at least the sample chamber of the system for monitoring pulp chemistry data is positioned on the plant site as close as possible to the process stream as to shorten the distance between sample point on the process stream and the sample chamber thereby minimizing change to the chemistry of the pulp. In fact, pumping the slurry of interest around can, through the ingress of atmospheric oxygen, affect the chemistry of the sample and this may influence the measurements and even may result in false measurements.

By measuring the pulp chemistry data in-situ in the sample chamber and analyzing the pulp chemistry data directly when the data are measured, the data are actually processed in real-time. Thus processed data can then be directly, i.e. real-time, be transmitted to the operator of the mineral processing plant or water processing plant, preferably to a operator interface element. So, according to the method of the invention, the operator can be informed during the operation of the plant process of the actual pulp chemistry data of the plant. In fact, the measuring, analyzing and transmitting of the pulp chemistry data can be said to occur online, i.e. during operation of the plant, and in real-time, i.e. the data are analyzed directly after measurement. This is a breakthrough with respect to the conventional method of ex-situ measuring and analyzing of the pulp chemistry data.

The interface element may be embodied in various ways, for example as an interface panel in an operating room, or as an application (app) on a smartphone, or as a computer display, or as a touchscreen, or as an interactive computer program, or as any other digital or analog display etc.

Measurement of the pulp chemistry data in the sample chamber can be done by probes known in the art that are able to measure and log the pH, Eh, dissolved oxygen, temperature, conductivity and/or oxygen demand. Before use in the sample chamber, the probes are calibrated. After each sample and measurement run, the probes are cleaned and prepared for the next run. Cleaning the probes is completed after the sample chamber is emptied. Usually, the sample chamber is flushed with water to clean the sample chamber itself, and the probes are sprayed with a jet of water to dislodge any build up thereby cleaning them.

Typically, the parameters of pH, Eh, dissolved oxygen, temperature, conductivity and/or oxygen demand are measured, as well as in a mineral processing plant as in a water treatment plant.

Preferably, the measurement run starts as slurry is introduced to the sample chamber. The slurry in the sample chamber may be stirred to keep the solids in the slurry suspended to obtain a representative, stable, homogeneous sample.

Alternatively, the measurement run may start once the sample chamber is full. Alternatively and/or additionally, measurement of at least the dissolved oxygen may start when the slurry is introduced in the sample chamber, while measurement of other parameters may start when the sample chamber is full, e.g. during stirring, or intermittent to the stirring, or after the stirring. The measurements of the respective probes are collected. An analysis tool that has logic that is capable of analyzing the measured data, such as a computer, or a chip, may then analyze the measured data and output the analyzed data. The analyzed data may then be transmitted to the operator interface element.

From the dissolved oxygen, the oxygen demand can be determined by using the following equation DO=DO₀·e^{-kt}; wherein DO is the dissolved oxygen at time t; DO₀ is the dissolved oxygen at time zero; k is the oxygen demand rate constant. A large k value suggests that the process has a high oxygen demand; a low k value suggests that the process has a low oxygen demand. The oxygen demand or the rate the pulp consumes oxygen is a measure for the reactivity of the mineral pulp of the mineral processing plant.

To collect the dissolved oxygen data, for use in the above equation to determine the oxygen demand, measurement thereof is commenced when the dissolved oxygen probe is still in air, and continues during filling of the sample chamber. The measurement of the dissolved oxygen data continues, during agitating of the slurry for a predetermined length of time, typically at least two minutes, Based on the thus collected measurements, the oxygen demand can be calculated using the above mentioned equation.

Advantageously, when emptying the sample chamber, the sampled slurry is returned to the process stream. By doing so, minimal loss of slurry can be obtained. Also, complex installations for handling and discharging the sampled slurry can be omitted. Since, the sample chamber is located on the plant site, the sampled slurry can relatively easily be returned to the process by providing a sample return line from the sample chamber to the process stream.

Since the slurry is continuously sampled from the process stream, there is a continuous flow from the sample point at the process stream to the system for monitoring pulp chemistry data (the PCM-system) comprising the sample chamber, e.g. via a sample feed line. The sample slurry discharges from the sample feed line into a sump that surrounds the sample chamber. When it is necessary to add the sample slurry to the sample chamber the sample feed line is directed to the sample chamber by a movable arm, e.g. a swiveling arm. In an embodiment, the arm may be actuated by a pneumatic piston. Once the sample chamber is full with sampled slurry, the swiveling arm directs the sample feed line back to the sump. In this way the slurry flow from the process stream of interest is continuous, and the possibility of blockages in the sample feed line are avoided. The excess slurry that bypassed by the sample chamber is collected in the sump and is returned to the process.

The frequency with which a sample is measured on the PCM-system, analyzed and the data are transmitted to the operator interface element may be up to 20 times per hour or possibly more, depending on the cycle time for a sample run. Preferably, the analyzed data are transmitted after each sample run. Typically, a sample run may take approximately 2 to 5 minutes, but this can be shortened or extended depending on the circumstances found in the operating plant. In an embodiment, the sample time is approximately 3.5 minutes, if data from two process streams are collected, the sample time is approximately 7 minutes, etc. Thus, analyzed data can now be transmitted almost immediately after the sample run, i.e. in real-time, contrary to the conventional laboratory method, wherein it could take a few days before the analyzed data would be available.

In another aspect of the invention, an additional sample slurry can be taken from the continuous process stream or from the PCM-system supply sample stream to perform an EDTA extraction. Performing EDTA extraction on a sample of slurry may give information on the oxidation state of the pulp. A slurry sample of a known volume is collected and deposited into a sample phial to which a 3 percent EDTA solution is added. The EDTA/slurry mixture is mixed for about 5 minutes before the solid and liquid phases are separated. The liquid and solid phases are typically separated by centrifuging. In an embodiment, the liquid phase may be additionally filtered after centrifuging. The liquid phase is analyzed using XRF (X-Ray Fluorescence). It is also possible to discharge the sample and any waste of the EDTA extraction process into the sump of the PCM-system and to return this material to the process as well. Performing analysis of the EDTA solution by means of XRF is advantageous as it is a relatively cheap and simple analysis that can occur in a short time frame and gives reliable results. The analysis of the EDTA solution can be performed online on an operating plant and providing data to the operation interface almost in real-time. Instead of XRF, other methods such as AAS, UV or other analytical methods can be used.

In an embodiment, a unit for performing EDTA extractions is provided, preferably as a separate module, that can be mounted to the PCM-system, that is arranged to perform the EDTA extraction, analysis and provide the data generated. In an other embodiment, the EDTA-extraction unit may be integrated to the PCM-system. The EDTA module can, if desired operate independently of the PCM-system as a bench scale analyzer.

Such an EDTA unit preferably comprises the components to perform the EDTA extraction. These may be: a pumping system to circulate slurry, a slurry sampling device to extract a known volume of slurry and inject it into the sample phial, an EDTA solution delivery system to add the correct volume of EDTA, a mixing system to mix the slurry and EDTA for approximately 5 minutes, a centrifuge to separate the solid phase form the liquid phase, a filter to the supernatant from the centrifuge, and a XRF device to analyze the EDTA solution. A control system supervises the processing of the slurry sample, and processes the measured data before presenting the data to an operator interface element.

Depending on the configuration the EDTA extraction module is able to receive slurry samples for analysis either continuously when coupled to the PCM-system or intermittently when employed as a bench scale instrument. In a preferred configuration the EDTA extraction unit can be positioned on the plant site as close as possible to the process stream as to shorten the distance between sample point on the process stream and the EDTA extraction module thereby minimizing change to the chemistry of the pulp. In an embodiment, the EDTA extraction unit can be provided as a module to the PCM-system. Also, by positioning the EDTA extraction module and performing the EDTA extraction on the plant site, the analysis can be done online on an operating plant and the thus generated data from the EDTA analysis can be returned to the operator interface element almost real-time. So, an operator of the plant almost has immediately feedback on the oxidation state of the minerals in the process of the operating plant and, when appropriate, may take actions to control the mineral process.

The method of performing the EDTA analysis may comprise the steps of taking a sample from the process stream; stirring the sample; performing extraction of the sample; using XRF to analyze the extracted solution; processing the data from the analysis and output the data to an operator interface element.

The method and unit for performing EDTA extraction may be considered as an invention in its own right.

Further advantageous embodiments are represented in the subclaims.

The invention further relates to a system for monitoring pulp chemistry data.

The invention also relates to a control system therefore and the invention further relates to a mineral or water processing plant comprising a system for monitoring pulp chemistry data.

The invention will further be elucidated on the basis of exemplary embodiments which are represented in a drawing. The exemplary embodiments are given by way of non-limitative illustration.

In the drawing:

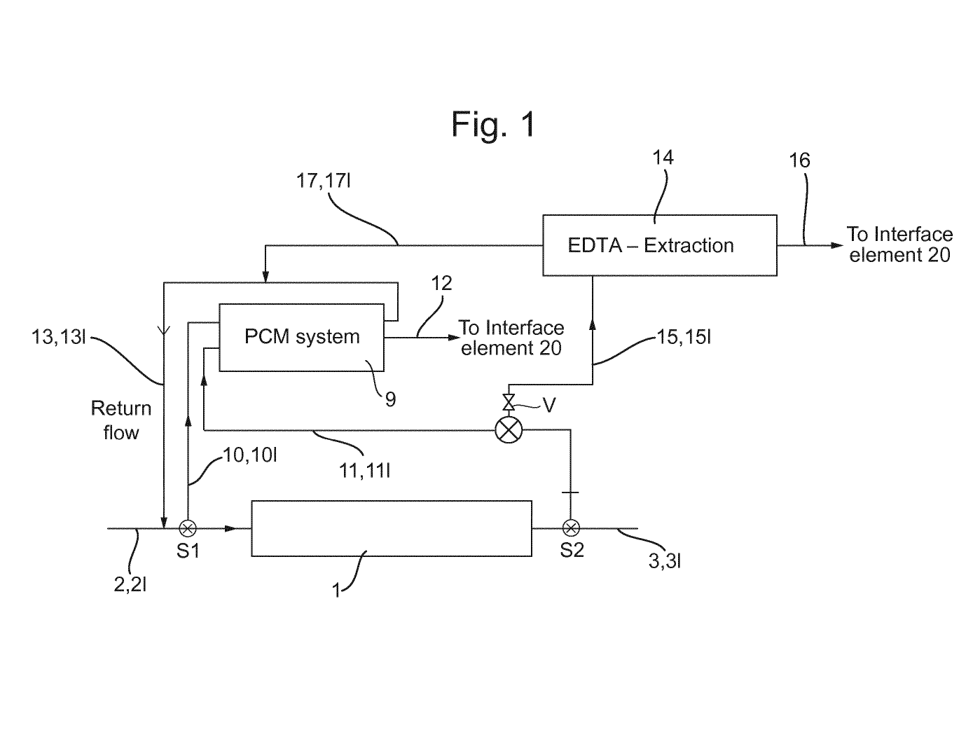

FIG. 1 shows a typical processing step of a mineral processing plant and/or a water treatment plant;

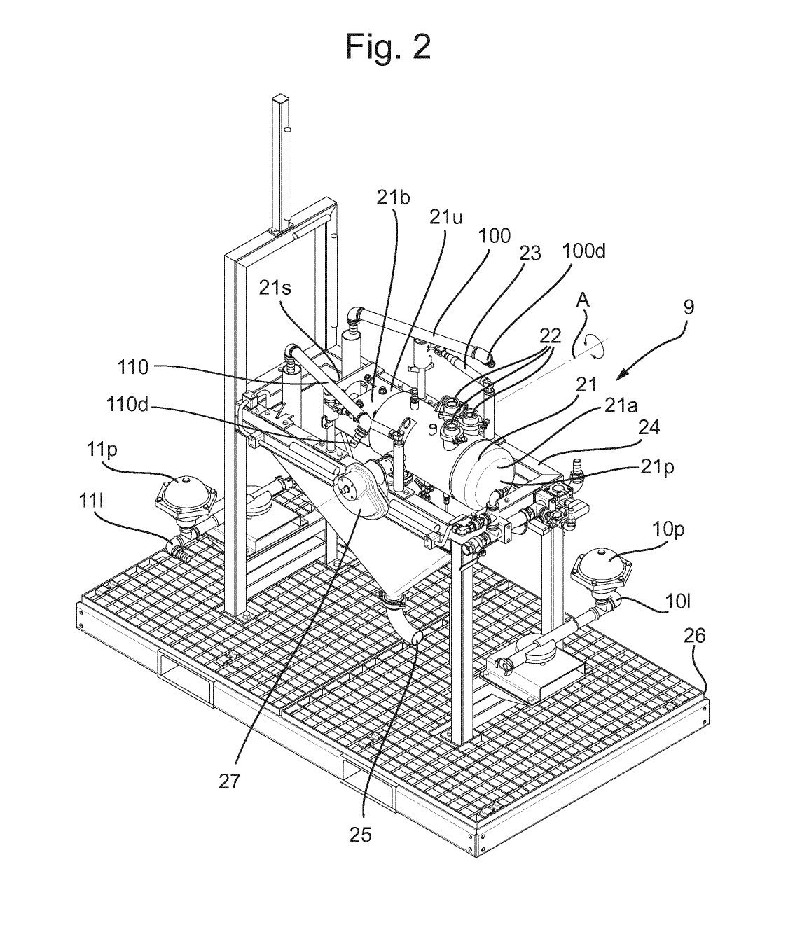

FIG. 2 shows an embodiment of a PCM-system;

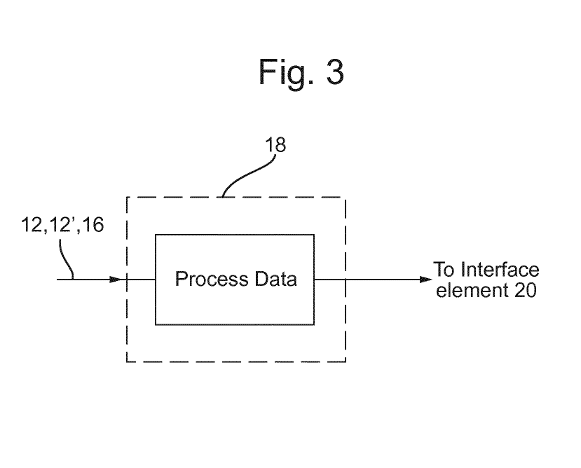

FIG. 3 shows schematically a process scheme of a control system for processing measured data;

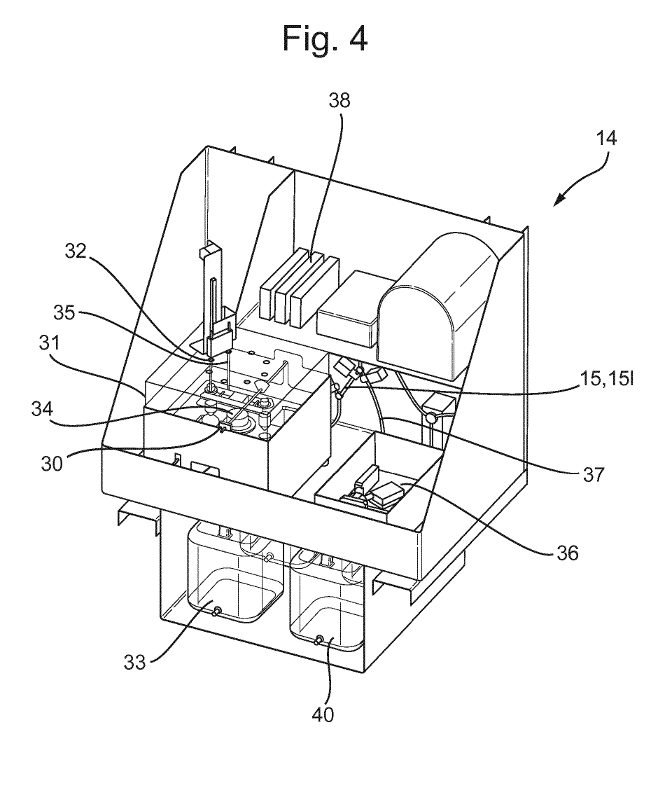

FIG. 4 shows schematically an EDTA extraction unit.

It is noted that the figures are only schematic representations of embodiments of the invention that are given by way of non-limiting example. In the figures, the same or corresponding parts are designated with the same reference numerals.

FIG. 1 shows an example of a typical process step 1 having a feed stream 2 and a tail stream 3. A process stream is fed to the process step 1 as a feed stream 2, then undergoes a treatment in the processing step 1, and exits from the processing step as a tailing stream 3. What treatment is given in the processing step is not essential for the invention. Such a process step 1 is typically part of a larger processing scheme for a mineral processing plant or a water processing plant. Such processing plants usually have multiple processing steps, each having a feed stream and a tailing stream, wherein the feed stream of one processing step may be the tailing stream of a previous processing step etc. In general, the processing schemes for mineral processing plants and/or water processing plants are known to the person skilled in the art. Also, the design and operation of such a mineral processing plant and/or water processing plant is well known to a person skilled in the art and will not be elaborated on in this document. According to the invention, a system for monitoring pulp chemistry data 9, also called a PCM-system, is provided.

Typically, as shown in FIG. 1, the PCM-system 9 is connected to a process feed stream line 21 through which the feed stream 2 flows, to the processing step 1 via a sample feed line 10l at a sample point S1. In this embodiment, the PCM-system 9 receives sample slurry in pairs, i.e. from a feed sample point S1 from a feed process stream 2 and a tailing sample point S2 from a tailing process stream 3 of a processing step. There are thus two sample feed lines 10l and 11l feeding the PCM-system 9 with feed sample flow 10 and tailing sample flow 11. The slurry from sampling points S1 and S2 flows continuously to the PCM-system 9. Multiple processing steps may be equipped with a PCM-system and/or a PCM-system may receive sample flow from multiple processing steps.

The PCM-system 9 is arranged for measuring and analyzing pulp chemistry data from the sample flows 10, 11. The analyzed pulp chemistry data from PCM-system 9 are provided to an operator interface element 20 on-line and in real-time. The PCM-system 9 comprises a data output 12 that outputs the analyzed pulp chemistry data to an operator interface element 20 of the mineral or water processing plant and/or of the processing step. In an embodiment each PCM-system 9 is provided with its respective operator interface element 20. In another embodiment, a single operator interface element 20 may be provided for representing the measured data of the PCM-systems 9.

During a measurement run of the PCM-system 9 data characterizing the pulp chemistry of the sampled slurry are measured and analyzed. After a measurement run, the sampled slurry is returned to the process stream, for example to the feed stream 2 by a return flow 13. Also, as the sample flow 10, 11 is continuously taken from the process stream, the slurry samples arriving at the PCM-system 9 are returned to the process via flow 13. As such, there is no or limited loss of pulp and almost all pulp can be processed through the mineral processing plant or water processing plant. Since the measurement runs of the PCM-system 9 are done batch-wise and the sample flow 10 and 11 are taken continuously from the process stream 2a the sample flow can be returned to the process stream 2a via the return flow line 13 during the batch measurement run.

This arrangement can be applied on every processing step of the mineral processing plant and/or of the water processing plant.

As the sample feed stream 10, 11 flows continuously to the PCM-system 9, it can be said to be running on-line, i.e. on an operating mineral or water processing plant, contrary to the conventional ex-situ laboratory systems. Also, the data determined by the PCM-system can be provided directly, or immediately, after a measurement run to the operator interface element. The PCM-system can thus be said to be running in real-time since there is almost immediate or direct feedback to the operator interface element after each measurement run. This is contrary to the conventional prior art ex-situ system, where it could take days before results of the laboratory measurements are available. By that time, the conditions on the operating mineral processing plant might have been changed already.

The measured data of the PCM-system 9 might be provided to the operate interface element 20 via wire transmission, or bus transmission, or wireless transmission, etc. Many communication systems may be used to transmit the data.

The operator interface element may be an interface such as a display mounted in the operator control room of the mineral processing plant 1. The display may be a computer display or a touchscreen display etc. The operator interface element may also be provided as an application (‘app’) on a mobile device such as a smartphone or a computer tablet or a laptop etc. The operator interface element such as a display, or a mobile device application may simply represent the measured information. To interpret the measured data, the operator might require his skills and knowledge. Also, the operator interface element may be accompanied by interpretation schemes, to help interpret the meaning of some of the measured values. Additionally, the operator interface element may not only provide the measured data, but may also be accompanied by a manual or application how an operator may act when certain values of certain parameters are measured. As such, a kind of open loop feedback may be provided to the operator. In a further step, the feedback may be provided closed loop and the process of the mineral processing plant or of the water processing plant may be influenced and/or adjusted depending on the measured data. Many variants of operator interface elements may be possible, as well as combinations of a static interface panel in e.g. the operator control room and mobile interface elements, e.g. an application on a mobile device may be possible.

According to the invention, the PCM system 9 has the capacity to collect samples from either one, two or more process streams such as e.g. in FIG. 1. The choice of sampling point(s) typically may vary depending on the application and the data requirements of the plant in question. In some instances only the feed to process may be sampled and analyzed, in others both the feed stream to and the tailing stream from the processing step may be sampled and analyzed. In other instances, multiple feed streams and/or tailing streams of multiple processing steps may be sampled and analyzed. The collection of the feed and tailing samples actually provides the opportunity to use the pulp chemical data in some form of process control strategy that may improve the stability of the process and ultimately lead to increased concentrate grades and/or mineral recoveries and/or improved water quality. For example, in a flotation mineral processing plant the most likely process streams to be sampled and analyzed are the rougher feed stream and rougher/scavenger tailing stream, and the first cleaner feed stream and the cleaner/scavenger tailing stream. However, it should be pointed out that it is possible to sample and analyze other process streams that may be critical to the process of the particular processing plant. For example, in a leaching operation the most likely process streams to be sampled and analyzed may be the leach feed stream and tailing stream. It is also possible to use the PCM-system to sample and analyze effluent water from a variety of systems using the same approach as described herewith. It is also to be recognized that more than one PCM-system may operate with in a plant.

Further, according to the invention, an EDTA-extraction unit 14 can be added to the PCM-system 9, or can be operated independently, as for example shown in FIG. 1. A subsample of the feed flow 15 to the PCM-system 9 is directed to the EDTA-extraction unit 14 In the example shown in FIG. 1, feed flow 15 is sampled from the PCM-system 9 feed flow line 11l from the tailing process stream 3. In a preferred embodiment, the feed flow 15 is sampled from the feed flow 10 from the feed process stream 2. Preferably, the feed flow 15 for the EDTA-extraction unit 14 is sampled continuously from the feed flow to the PCM-system. Alternatively, the feed flow 15 for the EDTA-extraction unit 14 is sampled intermittently or batch-wise from the feed flow to the PCM-system. In case of intermittent or batch-wise sampling, a valve V may be provided in the feed flow line 15 to open and close the feed flow line 15 to the EDTA-extraction unit 14.

The EDTA-extraction process is typically a batch process, so when the feed flow 15 is continuously sampled, the excess feed flow may be returned to the process stream of the mineral processing plant or water processing plant via a return flow line 17l. For example, the return flow 17 of the EDTA-extraction unit 14 may be discharged in the return flow line 13l, of the PCM-system 9 or may be discharged in the process stream line 21 of the process stream. Either variants or combinations thereof are possible.

The data 16 obtained from the EDTA-extraction of the sample is analyzed and provided to an operator interface element 20. Preferably, this is the same operator interface element that receives the data from the PCM measurements, however, it may be a separate or independent operator interface element. Idem as for the PCM-operator interface element, the EDTA operator interface element may be static, such as a display or a touchscreen in an operator control room, or may be an application (‘app’) on a mobile device, or combinations thereof. Also, the measured data from the EDTA-extraction unit may be accompanied by an interpretation manual and/or by a suggestion for interference for certain measured values as an open loop feedback, or may even be extended into a closed loop feedback.

As shown in FIG. 3, there is a control system 18 provided that processes the data measured in the PCM-system 9 and/or in the EDTA-extraction unit 14. There may be a single control system provided that is configured to process the measured data of all the PCM-systems on the mineral processing plant as well as of all the EDTA-extraction units 14 on the mineral processing plant or water processing plant. Alternatively and/or additionally, each PCM-system 9 and/or EDTA-extraction unit 14 may be provided with its dedicated control system 18. The control system 18 processes the measured data and provides the processed data to the operator interface element 20. Then, the data can be interpreted by the operator. Depending on the frequency of the measurement runs, the analyzed data may be provided to the operator interface element 20 up to twenty times per hour, which is a major advantage with respect to the off-line and ex-situ prior art methods.

An embodiment of the PCM-system 9 is shown in FIG. 2. The PCM-system 9 comprises two sample feed lines 10l, 11l that are connected to the process flow lines, here feed flow line 21 and tailing flow line 31, for example via a hose, as schematically represented in FIG. 2. In this embodiment there are two feed lines, alternatively a single feed line may be provided or more than two feed lines may be provided.

The feed lines 10l, 11l are connected to swivel arms 100, 110. The connection, for example via a flexible hose, is not shown in this figure, but can easily be established by connecting the flexible hose at one end to the feed line and at another end to an input end of the swivel arm. A pump 10p, 11p can be provided on the feed lines 10l, 11l to pump the slurry to the swivel arms 100, 110.

Further, a sample chamber 21 is provided in which the sample is collected and measurements are done. Thereto, the sample chamber 21 is provided with measurement probes 22. The probes 22 measure values of their respective parameters and offer these measured values to the control system 20 (not shown here). The control system 20 may be a computer arranged adjacent the PCM-system 9, or remote from the PCM-system 9, e.g. in the operator control room. Here, three probes 22 are provided, but in another embodiment a different number of probes may be provided.

The sample chamber 21 is here embodied as a cylindrical tank, but can have other shapes as well. The sample chamber 21 is rotatable around an axis A for filling and emptying of the sample chamber 21. The sample chamber 21 is mounted in a trough or sump 24. At a bottom end of the trough, a discharge 25 is provided. This discharge 25 can be connected with the process stream via a return flow line 13, for example a steel pipe or a flexible hose.

The sample chamber 21 is rotatable between a lying position and standing (upward and downward) positions around the axis A by a motor 27. The motor 27 can be an electric motor, or a pneumatic motor or a hydraulic motor, or can be a pneumatic or hydraulic cylinder. Any actuator can be used to rotate the tank 21.

The probes 22, are located in the side 21a of the chamber 21, but can be accessed from outside for easy removal and/or exchange. The top 21b of the sample chamber 21 is open. The open end 21b is provided to receive the slurry from either one of the swivel arms 100, 110 when the sample chamber 21 is in an upright position. Also, the slurry can be discharged from the sample chamber 21 via the open end 21b when the sample chamber 21 is in a downward position. The slurry is then discharged in the trough or sump 24.

To fill the sample chamber 21, the swivel arms 100 or 110 move across to the top 21b of the sample chamber 21 and slurry is discharged into the chamber 21 for a known time. At the end of this time the swivel arm 100, 110 returns to a position in which the slurry now bypasses the sample chamber 21. In the filling position the swiveling arm 100, 110 is above the open end 21b of the sample chamber 21 when the latter is in upward position. In the bypassing position, the swivel arm 100, 110 is aside of the sample chamber 21 and above the trough or sump 24 such that the slurry is discharged from the swiveling arms 100, 110 into the trough 24.

In this embodiment, the sample chamber 21 has a curved or hemispherical bottom part 21a and an open top part 21b. In the bottom part 21a the probes are provided. The open top part 21b allows for access to the sample chamber 21 for the agitator to stir the sample in the sample chamber 21, for the sample to be introduced via the open top part into the sample chamber, for removing the sample out of the sample chamber, for water jets to clean the sample chamber etc. Alternative embodiments of the sample chamber are possible.

For example, a closed top sample chamber can be provided having feed lines comprising a splitting station with a valve that may have an open mode for allowing the sample into the sample chamber and a closed mode for bypassing the sample of the sample chamber. Also, the sample chamber is preferably be provided with an agitator to stir the sample inside the chamber, the motor of the agitator is preferably mounted outside of the sample chamber. Further, an exit line may be provided allowing the sample to exit the sample chamber after a measurement run. In such an embodiment, water jets or water lines for cleaning the sample chamber after a measurement run may be provided inside of the sample chamber. Alternatively and/or additionally a water line may be provided that connects to the splitting station such that water is flushed through the sample inlet into the sample chamber, having the advantage of at the same time cleaning the sample inlet line. In an embodiment, the splitting station can be configured having two input lines, a sample line and a water line, and having two valves, one sample valve and one water valve, and having two output lines, one towards the sample chamber and one bypassing the sample chamber. In such an embodiment, rotation or tilting of the sample chamber may or may not be omitted. Also, in such an embodiment, instead of swiveling arms, the feed lines may be provided with a valve to allow for a permanent connection with the sample chamber and the swiveling arms may be omitted. Many variants of a sample chamber may be possible.

Further, the tank 21 can be provided with a motor for stirring the slurry in the sample chamber 21. The motor can be positioned at an end side 21s or at the upper side 21u. The motor is connected with a stirring arm or agitator of which the vanes typically extend near a bottom of the sample chamber 21 at an opposite end side 21p. Various embodiments for the motor are possible, electric, hydraulic, pneumatic, magnetic etc. Advantageously, the agitator is driven at relatively slow rotational speed to keep the solids suspended.

The swiveling arms 100, 110 can be adjusted by a pneumatic piston 23. When bypassing the sample chamber 21, the slurry is fed to the trough 24 that has a discharge 25 at its bottom end via which the slurry can be fed back to the process stream.

In an upright position, the tank 21 is rotated around 90 degrees such that the open end 21b is up and the end 21a is down. When the open end 21b is up, the swivel arm can move until a discharge end 100d, 110d of the swivel arm 100, 110 is moved above the open end 21b such that the slurry can be discharged in the tank 21. When the tank 21 is full, the tank 21 is rotated back to the lying approximately horizontal position in which the measurements can take place. When the measurement run is finished, the tank 21 can be rotated about 90 degrees in the other direction such that the open end 21b is down and the probes-end 21a is up. The sample can then be discharged from the tank 21 through the open end 21b. In the trough 24 further water sprays are provided to clean the inside of the tank 21 and flush the probes 22 after emptying the tank. Cleaning of the tank is preferably with water, and the probes 22 are flushed, preferably with water as well. When cleaned, the sample chamber 21 can be rotated back to the lying or horizontal position and further to the upward standing position to be filled again.

Typically, the measurement run starts when the slurry is introduced in the sample chamber 21, as to correctly measure the dissolved oxygen, thus starting when the dissolved oxygen probe is still in air. Other parameters such as pH, Eh, temperature, conductivity and/or oxygen demand may be measured via their respective probes. In the sample chamber 21, the slurry may be stirred to keep the solids in the slurry suspended to obtain a representative, stable, homogeneous sample.

After each measurement run, the sample chamber 21 is being emptied into the trough 24 and the slurry then is discharged via the discharge opening 25 and flow lines (not shown here) to the process stream.

Typically, the sample chamber 21 can alternately be filled by slurry from swivel arm 100 and by slurry from swivel arm 110. For example, the swivel arm 100 receives slurry from the feed stream of the process stream and the swivel arm 110 receives slurry from the tail stream. So, alternating, data about the feed stream and data about the tailing stream can be obtained and can be presented to the operator interface element 20. In another embodiment, a single feed line is possible, or three or more feed lines are possible for which a measurement run can be done alternately.

The PCM-system 9 is based on a tray 26, making it a compact unit that can easily positioned on a predetermined location on the plant site, preferably relatively close to the process stream to avoid long flow lines between the sample points S1, S2 on the process stream lines and the PCM-system, as to not disturb the slurry too much, since this may negatively affect the measurements.

A control panel comprising the control system 18 may be provided, e.g. mounted to the PCM-system 9. The control panel can also comprise an interface element via which a user can change settings and/or parameters. The interface element even may comprise a display or screen showing the measured data for each process stream. Via a connection, e.g. wireless, Ethernet, electrical, etc. can the control panel be in communication with the operator interface element 20 in the operator control room on which the measured data can be presented as well.

An example of an EDTA-extraction unit 14 is shown in FIG. 4. Slurry is provided to the EDTA-extraction unit 14 via the feed flow line 15l entering the EDTA-extraction unit 14 at a rear side thereof. The feed flow line 15l samples slurry from the process stream e.g. from the feed flow line 10l, 11l of the PCM-system 9. Due to the intermittent nature of the EDTA-extraction process, the slurry is preferably sampled in batches, so preferably, a valve is provided in the feed flow line 15l at or near the sampling point. Alternatively, continuous sampling is possible, but the excess slurry is being returned to the process stream via a return flow line.

The EDTA-extraction unit 14 comprises an EDTA module 31 in which the EDTA-solution is added to the slurry. The slurry is added to a beaker or sample phial (not shown here) via the flow line 15l and via a dispensing device 32. Once in the beaker, the EDTA solution, typically a 3% EDTA solution, is added to the slurry, and is being stirred. The EDTA-solution can be added via a dispensing device 32. Preferably, the EDTA-solution is added to the beaker via the flow line 15l, which gives the advantage that the flow lines are being flushed with the EDTA solution. Typically, the EDTA solution is kept in a reservoir 33 at or near the EDTA extraction unit 14, here underneath the EDTA module 31. The solution in the beaker is then centrifuged, e.g. by a motor 34 to separate the solid phase and the liquid phase. The liquid phase is then taken from the beaker, for example via a pipe 35 to transport it to the XRF-module 36. In an embodiment, the liquid phase can pass a filter to remove finer solid particles. Typically, centrifuging of the solution may take about 30 minutes or longer. After centrifuging and dispensing the liquid phase to the XRF module 36, the beaker can be cleaned and/or flushed, preferably with water. To that end, a water spray can be provided in the EDTA module 31. Also, the beaker can be emptied and/or cleaned by means of a rotating mechanism 30.

The XRF module 36 receives the clear liquor via flow lines 37 from the EDTA module box 31. In the XRF-module 36, an XRF analysis is performed on the clear liquid phase, the results thereof being processed by a control unit 38. The clear liquor and other waste can be collected in a reservoir 40. From the reservoir 40 the liquid and/or waste can be fed back to the process stream, e.g. via flow line 17l. Alternatively, the liquid and/or waste can be fed back directly to the process stream via a return flow line 17l. Typically, running the XRF analysis may take about 5 to about 15 minutes. Many variants are possible for the XRF-analysis. For example, multiple short runs on the same liquor can be possible of which the results are averaged, or a relatively long run is possible giving a more ‘stable’ result. Usually, it may be sufficient to perform a few times, e.g. three times, a relatively short run, of e.g. 5 minutes, and then to average the measured results. Instead of using the XRF-method, other methods such as AAS or UV could be used. However, XRF is preferably used in view of reliability and/or simplicity.

The results of the EDTA-extraction are typically known after 40 to 45 minutes from sampling, which is a major improvement with respect to prior art ex-situ methods. Having the results available from the EDTA-extraction in such a relatively fast way gives a major advantage to the process operator in operating the mineral processing plant or water processing plant. While running an XRF-analysis, the beaker of the EDTA-module 31 can be filled again with sample slurry for a next centrifuging run.

Further, a power supply and/or a control unit 38, such as a computer may be provided. The control unit 38 preferably is provided with a control system 18 to analyze and process the measured data and provide the analyzed data to the operator interface element.

For the purpose of clarity and a concise description, features are described herein as part of the same or separate embodiments, however, it will be appreciated that the scope of the invention may include embodiments having combinations of all or some of the features described. It may be understood that the embodiments shown have the same or similar components, apart from where they are described as being different.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

Many variants will be apparent to the person skilled in the art. All variants are understood to be comprised within the scope of the invention defined in the following claims.

1. Method of monitoring chemistry parameters from an operating mineral or water processing plant, comprising:
continuously sampling a flow of slurry from a process stream within the operating mineral or water processing plant;
filling a sample chamber located on the plant site with the sampled slurry;
measuring pulp chemistry data of the sampled slurry in the sample chamber;
analyzing the measured pulp chemistry data;
providing the analyzed pulp chemistry data to an operator interface element of the plant in real-time;
emptying the sample chamber and refilling the sample chamber with sampled slurry.

2. Method according to claim 1, wherein the sampled slurry of the sample chamber is returned to the process stream of the operating plant when emptying the sample chamber.

3. Method according to claim 1, wherein the sampled slurry is bypasses the sample chamber for being returned to the process stream when the sample chamber is full.

4. Method according to claim 1, wherein the pulp chemistry data are provided to the operator interface element up to 20 times per hour.

5. Method according to claim 1, further sampling the continuous slurry sample to extract a slurry sample for EDTA extraction.

6. Method according to claim 5, further analyzing the subsequent EDTA solution by means of XRF, AAS, UV or the like, and processing the EDTA extraction data.

7. Method according to claim 6, further comprising providing the EDTA extraction data to the operator interface element.

8. Method according to claim 1, wherein the slurry is sampled from multiple process streams of the operating mineral processing plant resulting in multiple sample slurry flows being fed to at least one sample chamber located on the plant site.

9. Method according to claim 8, wherein multiple sample slurry flows of a single process step are taken as pairs, such that one sample slurry is of the feed stream of the process step and one sample slurry is of the tail stream of the process step, wherein the feed and the tail sample slurry are fed to the same sample chamber to monitor the pulp chemistry data of the associated process step.

10. Method according to claim 1, wherein the analyzed pulp chemistry data are one of: pH, Eh, dissolved oxygen, temperature, conductivity, oxygen demand and pulp oxidation state.

11. System for monitoring pulp chemistry data of an operating mineral or water processing plant, comprising at least one sample point on a process stream of a processing step of the operating plant for continuously sampling slurry from the process stream, a sample chamber for receiving the sampled slurry and a feed line between the sample point and the sample chamber to feed the sampled slurry to the sample chamber, wherein the sample chamber is located on the plant site and is arranged for measuring pulp chemistry data of the sampled slurry, further comprising a control system for processing the measured data and providing the measured data to an operator interface element in real-time.

12. System according to claim 11, wherein the feed line is arranged to fill the sample chamber and, when the sample chamber is filled, to bypass the sample chamber to return the sampled slurry to the process stream.

13. System according to claim 12, wherein the feed line comprises a swivel arm that swivels between a filling position to fill the sample chamber and a bypass position to bypass the sample chamber when the sample chamber is filled.

14. System according to claim 11, wherein the sample chamber is emptied after every measurement cycle.

15. System according to claim 14, wherein the sample chamber is arranged for tipping over to empty the sample chamber.

16. System according to claim 11, further comprising a sump arranged beneath the sample chamber for collecting sample slurry bypassed and/or from the sample chamber for being returned to the process stream.

17. System according to claim 11, further comprising an EDTA extraction unit to perform EDTA extraction on a sample retrieved from the sample chamber and/or collected from a feed line to the PCM-system.

18. System according to claim 17, further comprising a control system for processing the EDTA extraction data and provide the data to an operator interface element.

19. Control system for processing measured pulp chemistry data and/or EDTA extraction data and providing the processed data to an operator interface element.

20. Unit for performing EDTA extraction on a sample, preferably provided as a separate module, said module comprising measurement instruments for measuring EDTA extraction data and comprising a control unit configured for processing the measured EDTA extraction data and for providing the processed data to an operator interface element.

21-22. (canceled)
